(19) United States
(12) Patent Application Publication
(10) Pub. No.: US 2008/0082071 A1
(43) Pub. Date: Apr. 3, 2008

(54) Absorbent articles with graphic variety

(75) Inventors: Kristi J. Bryant, Appleton, WI (US); Kellie M. Goodrich, Appleton, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/542,492

(22) Filed: Oct. 3, 2006

(51) Int. Cl. A61F 13/15

(52) U.S. Cl. 604/385.02

(57) ABSTRACT

A method of providing an array of disposable absorbent articles. The array is manufactured by or for the same business entity. The method includes providing an array of at least two different disposable articles. The array includes a first disposable article having a first outer cover; a first absorbent structure disposed on the first outer cover; and a first graphic disposed on the first outer cover. The first disposable article is enclosed in a first package. The array also includes a second disposable article having a second outer cover; a second absorbent structure disposed on the second outer cover; and a second graphic that is different than the first graphic disposed on the second outer cover.

### BACKGROUND OF THE INVENTION

The present invention is generally related to a method of providing an array of disposable absorbent articles having a variety of graphics. More specifically, the present invention relates to providing some graphics with limited availability and providing other graphics with non-limited availability.

Disposable absorbent articles have long been known as personal care hygiene products. Disposable absorbent articles include infant diapers, diaper pants, training pants, swim pants, adult incontinence pads and briefs, and women's sanitary pads. Such absorbent articles are designed and constructed to absorb and store both solid and liquid bodily excretions such as urine, feces, menstrual fluid, or blood.

Disposable absorbent articles may be produced in a wide range of sizes and configurations to fit the needs of users of various sizes with different developmental needs, and in varying use situations (e.g. nighttime vs. daytime). In the past, manufacturers of disposable absorbent articles provided disposable absorbent articles without graphics, or more recently, with a specific graphic or set of graphics for each size and configuration. Some users may desire specialty graphics which are available for a limited time; alternatively some users may desire to maintain a consistent graphic which does not change over time. Manufacturers have attempted to meet the desires of both groups by compromise, specifically, by providing one graphic or set of graphics and changing these after a relatively long duration, for example graphics that change once a year. Because of the complexity in manufacturing and difficulties in managing the supply chain of specialty products, manufacturers have not provided specialty or limited-availability graphics in addition to these base graphics.

Accordingly, there remains a need for a method of providing an array of disposable absorbent articles that offer specialty limited-availability graphics while also providing a non-limited or base graphic as well.

### SUMMARY OF THE INVENTION

One aspect of the present invention includes a method of providing an array of disposable absorbent articles. The array is manufactured by or for the same business entity. The method includes providing an array of at least two different disposable articles. The array includes a first disposable article having a first outer cover; a first absorbent structure disposed on the first outer cover; and a first graphic disposed on the first outer cover. The first disposable article is enclosed in a first package. The array also includes a second disposable article having a second outer cover; a second absorbent structure disposed on the second outer cover; and a second graphic that is different than the first graphic disposed on the second outer cover. The second disposable article is enclosed in a second package. Further, the first disposable article is provided during a first period of time, a second period of time, and a third period of time. The third period of time is after the second period of time and the second period of time is after the first period of time. The second disposable article is provided during the second period of time and the second disposable article is not provided during the first period of time and the third period of time. The first absorbent structure and the second absorbent structure are substantially identical.

A second aspect of the present invention includes a method of providing an array of disposable absorbent articles. The array is manufactured by or for the same business entity. The method includes providing an array of at least three different disposable articles. The array includes a first disposable article having a first outer cover; a first absorbent structure disposed on the first outer cover; and a first graphic disposed on the first outer cover. The first disposable article is enclosed in a first package. The array includes a second disposable article having a second outer cover; a second absorbent structure disposed on the second outer cover; and a second graphic disposed on the second outer cover. The second disposable article is enclosed in a second package. The array also includes a third disposable article having a third outer cover; a third absorbent structure disposed on the third outer cover; and a third graphic disposed on the third outer cover. The third disposable article is enclosed in a third package. Further, the first disposable article is provided during a first period of time, a second period of time, a third period of time, and a fourth period of time. The fourth period of time is after the third period of time, the third period of time is after the second period of time, and the second period of time is after the first period of time. The second disposable article is provided during the second period of time, and the second disposable article is not provided during the first period of time, the third period of time, and the fourth period of time. The third disposable article is provided during the third period of time, and the third disposable article is not provided during the first period of time, the second period of time, and the fourth period of time. The first graphic differs from the second and third graphic, and the second graphic differs from the third graphic. The first absorbent structure, the second absorbent structure, and the third absorbent structure are substantially identical.

A third aspect of the present invention includes an array of disposable absorbent articles. The array is manufactured by or for the same business entity. The array includes a first disposable article having a first outer cover; a first absorbent structure disposed on the first outer cover; and a first graphic disposed on the first outer cover. The first disposable article is enclosed in a first package. The array includes a second disposable article having a second outer cover; a second absorbent structure disposed on the second outer cover; and a second graphic disposed on the second outer cover. The second disposable article is enclosed in a second package. Further, the first disposable article is provided during a first period of time, a second period of time, and a third period of time. The third period of time is after the second period of time and the second period of time is after the first period of time. The second disposable article is provided during the second period of time, and the second disposable article is not provided during the first period of time, and the third period of time. The first graphic differs from the second graphic. The first absorbent structure and the second absorbent structure are substantially identical.

### BRIEF DESCRIPTION OF THE DRAWINGS

The present invention will be more fully understood and further advantages will become apparent when reference is made to the following detailed description of the invention and the accompanying drawings wherein like numerals represent like elements. The drawings are merely representative and are not intended to limit the scope of the appended claims.

FIG. 1 illustrates a plan view of a disposable absorbent article in an unfastened, stretched, and laid flat condition with the surface of the article that contacts the wearer's skin facing the viewer and with portions of the article partially cut away to show the underlying features;

FIG. 2 illustrates an array of disposable absorbent articles; and

FIG. 3 illustrates a second array of disposable absorbent articles.

### DETAILED DESCRIPTION OF THE INVENTION

The present disclosure of the invention will be expressed in terms of its various components, elements, constructions, configurations, arrangements, and other features that may also be individually or collectively referenced by the term, “aspect(s)” of the invention, or other similar terms. It is contemplated that the various forms of the disclosed invention may incorporate one or more of its various features and aspects, and that such features and aspects may be employed in any desired operative combination thereof.

When introducing elements of the present invention or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements. “Disposed”, “disposed on”, “disposed with”, “disposed at”, “disposed near”, and variations thereof are intended to mean that one element can be integral with another element, or that one element can be a separate structure bonded to or placed with or placed near another element.

The present invention is directed to a method of providing an array of disposable articles, the array of disposable articles being manufactured by or for the same business entity. The array includes at least two different disposable articles, one of which is only available for a limited period of time, the other of which is available for a longer period of time.

In various aspects of the present invention, the disposable article 20′, 20″, 20′″ of the array may be branded with the same trademark or with different trademarks from the same trademark owner. An “owner” is defined broadly to include separate divisions or subsidiaries of a parent company or business entity. Thus, if two companies are owned by a common business entity yet own different trademarks, then the trademarks are considered to have common ownership. The articles 20′, 20″, 20′″ may be marked directly or indirectly, for example, by marking the package which contains the articles 20′, 20″, 20′″. In one aspect of the invention, the first disposable article 20′ and the second disposable article 20″ are associated with a first commonly owned trademark. One example of a trademark owned by the assignee of this invention is “HUGGIES.” However, this particular mark is not critical to the present invention—any desired trademark may be used. For example, the first 20′ and the second 20″ disposable article may both be marked with the HUGGIES trademark.

In various other aspects of the present invention, the first disposable article 20′, the second disposable article 20″, and/or the third disposable article 20′″ are marked with a commonly owned second trademark. As another non-limiting example, the first 20′, second 20″, and/or third 20′″ disposable article may be marked with the SUPREME trademark.

The principles of the present invention can be incorporated into a series of any suitable disposable articles. Examples of such suitable articles include diapers, diaper-pants, training pants, and incontinence products. For ease of explanation, the description hereafter will be in terms of an array of diapers.

### Representative Disposable Article

The individual disposable articles that make up the array will be described in greater detail in the context of a single diaper. Typically, disposable articles are intended for limited use and are not intended to be laundered or otherwise cleaned for reuse. A disposable diaper, for example, is discarded after it has become soiled by the wearer.

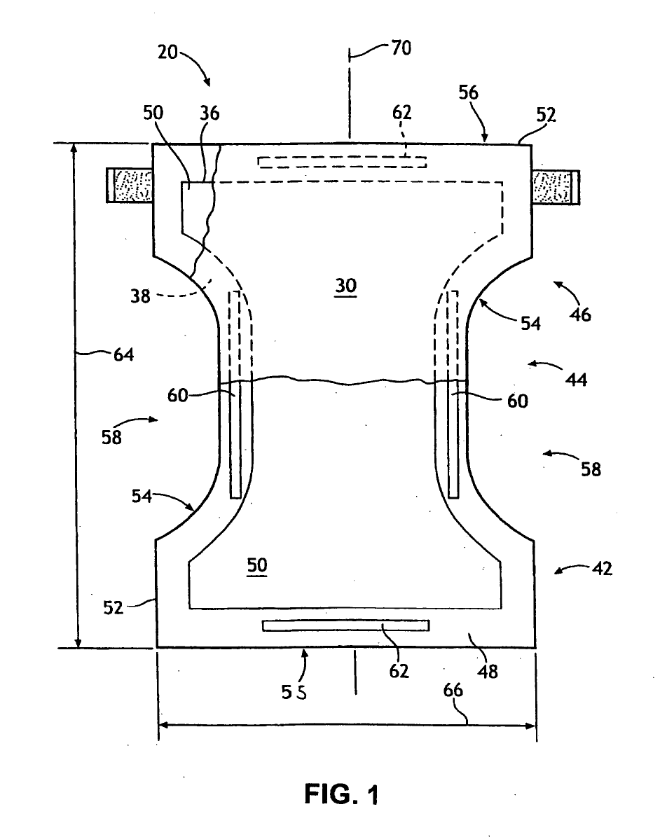

FIG. 1 illustrates a disposable diaper 20 which may be incorporated into the discussed arrays. The illustrated disposable diaper 20 has a front portion 42, a rear portion 46, and a crotch portion 44 located between the front and rear portions. The disposable diaper includes an outer cover 48, a bodyside liner 30, and an absorbent structure 50 situated between the outer cover 48 and the liner 30. The outer edges of the diaper 20 define a periphery 52 with laterally opposed, longitudinally extending side edges 54; longitudinally opposed, laterally extending end edges, specifically a front end edge 55, and a rear end edge 56; and a system of elastomeric gathering members, such as a system including leg elastics 60 and waist elastics 62. The longitudinal side edges 54 define a pair of leg openings 58 for the diaper 20, and optionally, are curvilinear and contoured. The lateral end edges 55, 56 are illustrated as straight, but optionally, may be curvilinear. The diaper 20 may also include additional components to assist in the acquisition, distribution, and storage of bodily exudates. For example, the diaper 20 may include a transport layer, such as described in U.S. Pat. No. 4,798,603, to Meyer et al., or a surge management layer, such as described in European Patent Office Publication No. 0539703, published May 5, 1993.

With regard to the designated surfaces of the absorbent article and its components, the various upper or bodyside surfaces are configured to face toward the body of the wearer when the absorbent article is worn by the wearer for ordinary use. The various opposing or lower surfaces are configured to face away from the wearer's body when the absorbent article is worn by the wearer.

The diaper 20 generally defines a longitudinally extending length dimension 64 and a laterally extending width dimension 66, as representatively illustrated in FIG. 1.

The outer cover 48 and the liner 30 may be generally coextensive (e.g., FIG. 1) or, optionally, may be non-coextensive. Either or both of the outer cover 48 and the liner 30 may have length and width dimensions that are generally larger than those of the absorbent structure 50 and extend beyond the corresponding dimensions of the absorbent structure 50 to provide longitudinal side edges 54 and lateral end edges 55, 56 which may be connected or otherwise associated together in an operable manner. As used herein when describing the liner 30 in relation to the outer cover 48 and vice versa, the term “associated” encompasses configurations in which the liner 30 is directly joined to the outer cover 48 and configurations where the liner 30 is indirectly joined to the outer cover 48 by affixing portions of the liner 30 to intermediate members which in turn are affixed to at least portions of the outer cover 48. The liner 30 and the outer cover 48 can, for example, be joined to each other in at least a portion of the diaper periphery 52 by attachment mechanisms (not shown) such as adhesive bonds, sonic bonds, thermal bonds, pinning, stitching, or a variety of other attachment techniques known in the art, as well as combinations thereof.

Various woven and nonwoven fabrics may be used for the liner 30. For example, the liner 30 may be composed of a meltblown or spunbonded web of polyolefin fibers. The liner 30 may also be a bonded-carded web composed of natural and/or synthetic fibers. The liner 30 may be composed of a substantially hydrophobic material, and the hydrophobic material may, optionally, be treated with a surfactant, or otherwise processed, to impart a desired level of wettability and hydrophilicity. Specifically, the liner 30 may be a nonwoven, spunbond, polypropylene fabric composed of about 2.8 to about 3.2 denier fibers formed into a web having a basis weight of about 22 gsm and a density of about 0.06 g/cc.

The liner 30 may also be surface treated with about 0.3 weight percent of a surfactant mixture that contains a mixture of AHCOVEL Base N-62 surfactant and GLUCOPON 220UP surfactant in about a 3:1 ratio based on a total weight of the surfactant mixture. The AHCOVEL Base N-62 surfactant is purchased from Hodgson Textile Chemicals Inc., a business having offices in Mount Holly, N.C., and includes a blend of hydrogenated ethoxylated castor oil and sorbitan monooleate in a 55:45 weight ratio. The GLUCOPON 220UP surfactant is purchased from Henkel Corporation, Gulph Mills, Pa., and includes alkyl polyglycoside. The surfactant may also include additional ingredients such as aloe. The surfactant may be applied by any conventional means, such as spraying, printing, brush coating, foam, or the like. The surfactant may be applied to the entire liner 30 or may be selectively applied to particular sections of the liner 30, such as the medial section along the longitudinal centerline of a diaper, to provide greater wettability of such sections.

The outer cover 48 may suitably be composed of a material which is either liquid permeable or liquid impermeable. It is generally desirable that the outer cover 48 be formed from a material which is substantially liquid-impermeable. For example, a typical outer cover 48 can be manufactured from a thin plastic film or other flexible liquid impermeable material. For example, the outer cover 48 may be formed from a polyethylene film having a thickness of from about 0.012 mm (0.5 mil) to about 0.051 mm (2.0 mils). If desirous of presenting the outer cover 48 with a more cloth-like feel, the outer cover 48 may include a polyethylene film having laminated to the lower or outer surface thereof a nonwoven web, such as a spunbond web of polyolefin fibers. For example, a polyethylene film having a thickness of about 0.015 mm (0.6 mil) may have thermally laminated thereto a spunbond web of polyolefin fibers, which fibers have a thickness of about 1.5 to about 2.5 denier per filament, which nonwoven web has a basis weight of about 24 gsm (0.7 osy). Methods of forming such cloth-like outer covers are known to those skilled in the art.

Further, the outer cover 48 may be formed of a woven or nonwoven fibrous web layer which has been totally or partially constructed or treated to impart a desired level of liquid impermeability to selected regions that are adjacent or proximate the absorbent structure 50. Still further, the outer cover 48 may optionally be composed of micro-porous “breathable” material, which permits vapors to escape from the absorbent structure 50 while still preventing liquid exudates from passing through the outer cover 48.

The absorbent structure 50 may include a matrix of hydrophilic fibers, such as a web of cellulosic fluff, mixed with particles of a high-absorbency material commonly known as superabsorbent material. In a particular version, the absorbent structure 50 includes a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed. One suitable type of wood pulp fluff is identified with the trade designation CR-1654, available from Bowater, Inc., Greenville, S.C., and is a bleached, highly-absorbent sulfate wood pulp containing primarily soft wood fibers. A special densification pulp, identified with the trade designation ND-416, available from Weyerhaeuser of Federal Way, Wash., is also suitable for use.

To limit any undesired movement of superabsorbent material, the disposable diaper 20 may also include a wrap sheet (not shown) which is placed immediately adjacent and partially or totally around the entire absorbent structure 50, around an individual layer of the absorbent structure 50, or around one or more selected elements of the absorbent structure 50, as desired. The wrap sheet is typically a layer of absorbent material that covers at least the upper and lower surfaces of the absorbent structure 50 prior to the absorbent structure 50 being situated between the outer cover 48 and the liner 30.

The absorbent structure 50 may have any of a number of shapes. For example, the absorbent structure 50 may be rectangular, I-shaped, or T-shaped. It is generally desired that the absorbent structure 50 be narrower in the crotch portion than the rear or front portion(s).

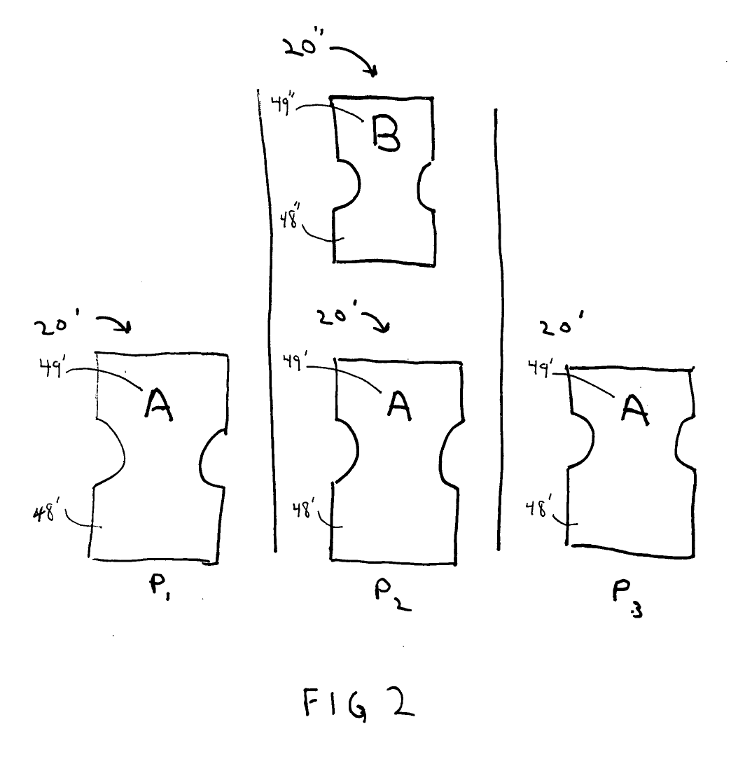

Referring to FIG. 2, an array of diapers is illustrated. The array includes two diapers 20, but an array of diapers in accordance with the present invention can include as many different diapers as necessary to provide the desired graphic variety. The array illustrates diapers which are provided at different periods of time. As illustrated the vertical columns represent diapers provided at a given period of time. As viewed from left to right, the first column P₁ illustrates a single first diaper 20′ provided during a first period of time. The second column P₂, illustrates the first diaper 20′ and a second diaper 20″ provided during a second period or time. The third column P₃ illustrates the first diaper 20′ provided during a third period or time. The second diaper 20″ is not provided during the first period of time P₁ or the third period of time P₃. As used herein, the third period of time P₃ is after the second period of time P₂ and the second period of time P₂ is after the first period of time P₁. The periods of time P₁, P₂, P₃ are distinct in that they do not overlap.

The availability or lack of availability of the first diaper 20′ and the second diaper 20″ provides several benefits. The availability of the first diaper 20′ during the first, second and third period of times P₁, P₂, P₃ allows retailers the choice to stock only the first diaper 20′. Retailers with limited available space for disposable articles, or retailers that sell few disposable articles may be hesitant to stock a specialty, limited-availability diaper. The availability of the first diaper 20′ for all time period allows these small retailers to minimize shelf resets. Alternatively, retailers that specialize in seasonal items, such as Halloween, Thanksgiving, or Independence day merchandise may choose to stock specialty, limited-availability diapers, such as the second diaper 20″, suitable for particular time periods either alone or in combination with the first diaper 20′.

The first diaper 20′ of the array has a first outer cover 48′ having an interior surface and an opposite exterior surface. The first diaper 20′ has a first absorbent structure disposed on the interior surface of the first outer cover 48′. The first diaper 20′ has a first graphic 49′ disposed on the first outer cover 48′. The first diaper 20′ may be enclosed in a first package.

The second diaper 20″ of the array has a second outer cover 48″ having an interior surface and an opposite exterior surface. The second diaper 20″ has a second absorbent structure disposed on the interior surface of the second outer cover 48″. The second diaper 20″ has a second graphic 49″ disposed on the second outer cover 48″. The second diaper 20″ may be enclosed in a second package.

To provide end users with both the ability to have consistent graphics as well as the option to have specialty, limited-availability graphics, the first graphic 49′ differs from the second graphic 49″. The difference between the first graphic 49′ and the second graphic 49″ will be described in greater detail below. The first diaper 20′ and the second diaper 20″ are adapted to fit the same user, consequently, the first absorbent structure and the second absorbent structure are substantially identical. For example, the first absorbent structure and the second absorbent structure may have similar lengths, similar widths, similar shapes, and similar amounts of absorbent material. The first diaper and the second diaper may differ only in graphics to provide improved manufacturing efficiency.

The periods of time P₁, P₂, P₃ may have any duration as suitable. The periods of time P₁, P₂, P₃ may all have the same duration. For example the first, second, and third periods of time P₁, P₂, P₃ may all have a duration of at least one month. Alternatively, the periods of time P₁, P₂, P₃ may have durations that are different. For example, the first period of time P₁, may have a duration of one month, the second period of time P₂ may have a duration of three months, and the third period of time P₃ may have a duration of two months.

The graphics 49′, 49″, 49′″ are suitably disposed on the outer cover. The term “disposed on” and variations thereof are intended to mean that one element can be integral with another element, or that one element can be a separate structure bonded to or placed with or placed near another element. Thus, the graphics can be formed or applied directly or indirectly to a surface of the outer cover, formed or applied between layers of a multiple layer outer cover, formed or applied to a substrate that is placed with or near the outer cover, formed or applied within a layer of the outer cover or another substrate, or other variations or combinations thereof. In particular embodiments, the graphics can be printed, sprayed, or otherwise applied directly on a layer of the outer cover. In other embodiments, the graphics can be applied to a layer placed with or near the outer cover, such as a substrate associated with the absorbent assembly, including but not limited to tissue layers, liquid handling layers, absorbent layers, or the like.

The graphics can be located on the exterior surface of the outer cover to enhance the visual impact of the graphics. Alternatively, however, the graphics can be located on the interior surface of the outer cover or between layers of a multilayer outer cover, provided the graphics remain visible from the exterior of the product.

The graphics can be formed on or applied to the outer cover or another substrate bonded to or placed with or placed near the outer cover by any suitable technique. The graphics are desirably registered with other components of the absorbent article during manufacture such that the graphics are positioned in the desired regions of the product.

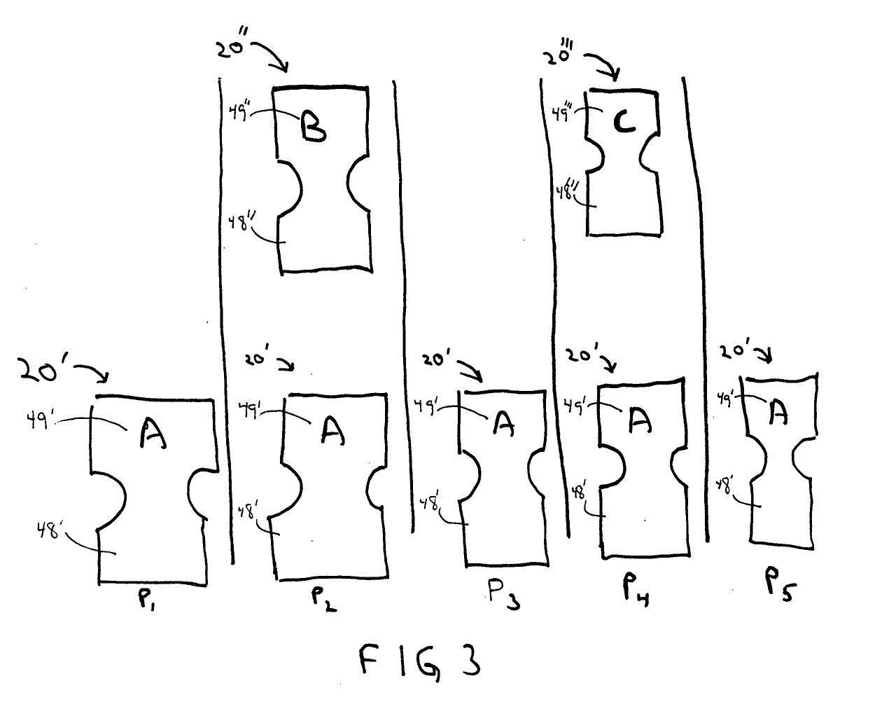

As shown in FIGS. 2 and 3, the diapers 20′, 20″, 20′″ and in particular the outer cover 48, desirably comprise one or more graphics. Examples of graphics include, but are not limited to: object graphics; character graphics; highlighting or emphasizing leg and waist openings in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label or a front label in the product; and registering written instructions at a desired location in the product.

The graphics of the absorbent article can be constructed to provide a story line involving a character graphic and an object graphic. The term “character graphic” is used herein to refer to a graphic containing an anthropomorphous image, and in particular an image having or suggesting human form or appearance which ascribes human motivations, characteristics or behavior to inanimate objects, animals, natural phenomena, cartoon characters, or the like. The character graphic may be suitable for children's underwear. The character graphics may be associated with popular characters in the media or advertising, or characters well known in a particular culture. Ideally they are characters that the child or caregiver care about and want to identify with. Ideally the child can imagine himself or herself taking the place of the character.

Suitable character graphics can include animals, people, inanimate objects, natural phenomena, cartoon characters, or the like that may be provided with human features such as arms, legs, facial features, or the like. It may be desirable for the character graphic to be familiar to the child, such as an identifiable cartoon character. The character graphics should at least be a type that the child can relate to, examples of which could include animals, toys, licensed characters, or the like. Character graphics can be made more personable and friendly to the child by including human-like features, expressions, apparel, abilities, or the like. By way of illustration, an animal character graphic can be shown smiling, wearing clothing, playing sports, fishing, driving, playing with toys, or the like. In particular embodiments, the character graphic can desirably be created to project an appearance that could be described as friendly, positive, non-intimidating, silly, independent, inspirational, active, expressive, dauntless, and/or persevering.

The term “object graphic” is used herein to refer to a graphic representing an object or thing, which can include an inanimate object or a character.

The object graphic may be interactively interrelated with the character graphic. As used herein, the term “interactively interrelated” is used to mean that the character graphic is illustrated to be involved in or performing an action or activity, and the object graphic is the object of or is associated with the action or activity. Without wishing to be limited to the specific embodiments listed, suitable examples of a character graphic interactively interrelated with an object graphic can include: the character graphic holding or using a racquet, bat, glove, other sporting equipment or the like and the object graphics comprising balls, related sporting equipment or the like; the character graphic illustrated on a soccer field, football field, basketball court, or like environment and the object graphics comprising balls, related equipment, or toys; the character graphic holding a butterfly net or the like and the object graphics comprising butterflies or the like; the character graphic holding a fishing pole, sitting in a boat or the like and the object graphics comprising fish, inflatable water toys or the like; the character graphic holding flowers, plants, gardening tools, or the like and the object graphics comprising flowers or plants; the character graphic feeding, training or nurturing the object graphics which comprise animals, plants, or the like; the character graphic playing in a specific environment such as a doll house, barn yard, or the like and the object graphics comprising dolls, animals, or the like which are specifically adapted to the environment; the character graphic holding or using a telescope or the like and the object graphics comprising stars, planets or the like; the character graphic comprising a racecar and the object graphic comprising race flags; the character graphic comprising a submarine and the object graphic comprising fish, bubbles, shells, or the like; the character graphic comprising a pet or other animal and the object graphics comprising dog bones, pet toys, food, or the like; the character graphic comprising a large sandbox and the object graphic comprising toys in the sandbox; the character graphic comprising a dog near a large dog dish and the object graphic comprising the dog bones inside or near the dog dish; or other suitable interactively interrelated graphics.

As used herein, the phrase “related in subject matter” refers to the situation where the subject matter of one graphic is the same as or is linked to the subject matter of another graphic. The subject matter relationship can be between two or more text messages, between two or more pictorial images, or between a combination of one or more text messages and one or more pictorial images. The term “text message” means a graphic consisting of one or more alphanumeric symbols, and the term “pictorial image” means a graphic consisting of one or more pictures. The terms “text image” and “pictorial image” are mutually exclusive as used herein. The general term “graphic” is used herein to mean any design, pattern, or the like that is or becomes visible on an absorbent article, and specifically includes text messages that consist of one or more alphanumeric symbols, pictorial images that consist of one or more pictures, and combinations thereof.

By way of example, two pictorial images are considered related in subject matter where the images are identical; separately illustrate different sizes, shapes, colors of a common object; each illustrate one and the other of two objects that are commonly associated with one another, such as the moon and stars, a body of water and water toys, a sandbox and suitable toys, a baseball bat and ball, a barn and animals, or the like; illustrate different items used in a particular activity, such as a sporting activity, a gardening activity or the like; jointly illustrate geometrically mating or engaging elements such as a triangle and a triangularly-shaped aperture, or two halves of a zipper; each illustrate one part of a multipart picture; or the like. Similarly, two text messages are considered related in subject matter where the messages: are identical; jointly form a sentence, thought, or action such as “jump” and “up”; each refer to one and the other of two items that are commonly associated with one another, such as “bat” and “ball,” “Big” and “Kid,” “Big” and “Girl,” or “Big” and “Boy”; jointly present a question and answer; or the like. Likewise, a text message and a pictorial image are considered to be related in subject matter where the text names, defines or describes the image; or the like.

The object graphic can be interactively related to the character graphic. Hence, in particular embodiments, an absorbent article includes an outer cover having an interior surface and an opposite exterior surface, and an absorbent assembly disposed on the interior surface of the outer cover. A character graphic and an object graphic are each disposed on the outer cover. The character graphic is interactively interrelated with the object graphic.

More particularly, the graphics of the absorbent article can include a character graphic and one or more object graphics which are related by a common story line, which would be generally known through books, movies, television programs, or other media sources. Further, the character graphic is involved in an activity utilizing the object graphics. For example, the graphics can include a character graphic consisting of an animal having human clothing, features, and abilities engaged in an activity such as fishing using a fishing pole, which is typically an activity undertaken by humans. The object graphics can comprise objects such as a boat, a lake, and a fishing pole which are associated with and/or define the activity of fishing.

The object graphic can alternatively be interactively unrelated to the character graphic. For example, the object graphic can comprise background graphics that are unrelated to the activity of the character graphic. By way of illustration, the object graphics can comprise images of the sky, the sun, grass, or the like.

The first graphic 49′ and the second graphic 49″ may contain any of the elements described above, in any combination. For example the first graphic 49′ may include a character graphic and an object graphic. The second graphic 49″ may also include a character graphic and an object graphic. In some arrays the character graphic of the first graphic 49′ may depict the same character as the character graphic of the second graphic 49″. For example the character graphic of the first graphic 49′ and the second graphic 49″ may be a dog. This commonality between the limited-availability second diaper 20″ and the non-limited-availability first diaper 20′ may provide the user with confidence that the first and second diapers 20′, 20″ will perform comparably.

The second graphic 49″ may contain a unique or specialty graphic. An example of a specialty graphic maybe a seasonal graphic. Seasonal graphics may relate to one of winter, spring, summer, or fall. For example, the seasonal graphics may illustrate snow in winter, falling leaves in fall, or budding flowers in spring.

The second graphic 49″ may contain seasonal graphics which relate to holidays, for example Mother's Day, Father's Day, May Day, Mardi Gras, Juneteenth, Spring Equinox, Chinese New Year, First Harvest, Carnival, Kwanza, Summerfest, Festival of Lights, King's Day, Queen's Day, Valentine's Day, St. Patrick's Day, Easter, Independence Day, Halloween, Thanksgiving, Christmas, Hanukkah, or New Years Day.

Information regarding the availability of the first diaper 20′ and the second diaper 20″ may be made available to consumers contemplating the purchase of one of the diapers in the array. Modes of conveying information to the caregiver may include in-store displays, posters, computer programs, brochures, package literature, shelf information, videos, information on the back of coupons, or any other suitable form of communication. The information could be available at stores, on television, in computer-friendly form, in advertisements, or through any other appropriate venue. The information may be included on the package containing the first diaper 20′, the package containing the second diaper 20″, or the package containing the first diaper 20′ and the package containing the second diaper 20″.

Referring to FIG. 3, a second array of diapers is illustrated. The array includes three diapers 20. The array illustrates diapers which are provided at different periods of time. As illustrated the vertical columns represent diapers provided at a given period of time. As viewed from left to right, the first column P₁ illustrates a single first diaper 20′ provided during a first period of time. The second column P₂, illustrates the first diaper 20′ and a second diaper 20″ provided during a second period of time. The third column P₃ illustrates the first diaper 20′ provided during a third period of time.

The fourth column P₄ illustrates the first diaper 20′ and a third diaper 20′″ provided during a fourth period of time. The fifth column P₅ illustrates the first diaper 20′ provided during a fifth period of time. The second diaper 20″ is not provided during the first period of time P₁, the third period of time P₃, the fourth period of time P₄, or the fifth period of time P₅. The third diaper 20′″ is not provided during the first period of time P₁, the second period of time P₂, the third period of time P₃, or the fifth period of time P₅.

As used herein, the fifth period of time P₅ is after the fourth period of time P₄, the fourth period of time P₄ is after the third period of time P₃, the third period of time P₃ is after the second period of time P₂, and the second period of time P₂ is after the first period of time P₁. The periods of time P₁, P₂, P₃, P₄, and P₅ are distinct in that they the do not overlap.

The availability or lack of availability of the first diaper 20′, the second diaper 20″, and the third diaper 20′″ provides several benefits. The availability of the first diaper 20′ during the first, second, third, fourth, and fifth periods of time P₁, P₂, P₃, P₄, P₅ allows retailers the choice to stock only the first diaper 20′. Retailers with limited space available for disposable articles or retailers that sell few disposable articles may be hesitant to stock a specialty, limited-availability diaper. The availability of the first diaper 20′ for all time periods allows these small retailers to minimize shelf resets. Alternatively, retailers that specialize in seasonal items, such as Halloween, Thanksgiving, or Independence Day merchandise, may choose to stock specialty, limited-availability diapers suitable for particular time periods such as the second diaper 20″ and/or the third diaper 20′″.

The first diaper 20′ of the array has a first outer cover 48′ having an interior surface and an opposite exterior surface. The first diaper 20′ has a first absorbent structure disposed on the interior surface of the first outer cover 48′. The first diaper 20′ has a first graphic 49′ disposed on the first outer cover 48′. The first diaper 20′ may be enclosed in a first package.

The second diaper 20″ of the array has a second outer cover 48″ having an interior surface and an opposite exterior surface. The second diaper 20″ has a second absorbent structure disposed on the interior surface of the second outer cover 48″. The second diaper 20″ has a second graphic 49″ disposed on the second outer cover 48″. The second diaper 20″ may be enclosed in a second package.

The third diaper 20′″ of the array has a third outer cover 48′″ having an interior surface and an opposite exterior surface. The third diaper 20′″ has a third absorbent structure disposed on the interior surface of the third outer cover 48′″. The third diaper 20′″ has a third graphic 49′″ disposed on the third outer cover 48′″. The third diaper 20′″ may be enclosed in a third package.

To provide end users with both the ability to have consistent graphics as well as the option to have specialty, limited-availability graphics, the first graphic 49′ differs from the second graphic 49″ and the third graphic 49′″, and the second graphic 49″ differs from the third graphic 49′″. The differences between the diapers of this second array, illustrated in FIG. 3, may correspond to the difference as describes above with respect to the first array, illustrated in FIG. 2. The first diaper 20′, the second diaper 20″, and the third diaper 20′″ are adapted to fit the same user, consequently the first absorbent structure, the second absorbent structure, and the third absorbent structure are substantially identical. For example, the first absorbent structure, the second absorbent structure, and the third absorbent structure may have similar lengths, similar widths, similar shapes, and similar amounts of absorbent material. The first diaper, the second diaper, and the third diaper may differ only in graphics. The first graphic 49′, second graphic 49″, and the third graphic 49′″ may contain any of the graphic elements as described above.

The periods of time P₁, P₂, P₃, P₄, P₅ may have any duration as suitable. The periods of time P₁, P₂, P₃, P₄, P₅ may all have the same duration. For example the first, second, third, fourth, and fifth periods of time P₁, P₂, P₃, P₄, P₅ may all have a duration of at least one month. Alternatively, the periods of time P₁, P₂, P₃, P₄, P₅ may have durations that are different. For example, the first period time P₁, may have a duration of one month, the second period of time P₂ may have a duration of three months, the third period of time P₃ may have a duration of two months, the fourth period of time P₄ may have a duration of six weeks, and the fifth period of time P₅ may have a duration of four months. The periods of time P₁, P₂, P₃, P₄, P₅ may have any combined duration of time as suitable for the array, for example the periods of time P₁, P₂, P₃, P₄, P₅ may have a combined duration of time of four months, six months, nine months, or a year.

In an alternative embodiment with a first, second, and third diaper 20′, 20″, 20′″, there may be four periods of time, P₁, P₂, P₄, P₅ such that a first limited-availability diaper (20″) is substituted with a second limited-availability diaper (20′″) without a period of time between the availability of the second diaper 20″ and third diaper 20′″.

As various changes could be made in the above constructions without departing from the scope of the invention, it is intended that all matter contained in the above description or shown in the accompanying drawings shall be interpreted as illustrative and not in a limiting sense.

We claim:

1. A method of providing an array of disposable absorbent articles, the array manufactured by or for the same business entity, comprising:
providing an array of at least two different disposable articles, the array comprising:
a first disposable article having a first outer cover; a first absorbent structure disposed on the first outer cover; and a first graphic disposed on the first outer cover; the first disposable article being enclosed in a first package;
a second disposable article having a second outer cover; a second absorbent structure disposed on the second outer cover; and a second graphic that is different than the first graphic disposed on the second outer cover; the second disposable article being enclosed in a second package;
wherein the first disposable article is provided during a first period of time, a second period of time, and a third period of time, the third period of time being after the second period of time and the second period of time being after the first period of time;
the second disposable article is provided during the second period of time, the second disposable article is not provided during the first period of time and the third period of time; and
the first absorbent structure and the second absorbent structure are substantially identical.

2. The method of claim 1 wherein the first period of time has a duration of at least one month, the second period of time has a duration of at least one month, and the third period of time has a duration of at least one month.

3. The method of claim 1 wherein the first absorbent article is marked with a trademark and the second absorbent article is marked with the same trademark.

4. The method of claim 1 wherein the first absorbent article is marked with a first trademark and a second trademark and the second absorbent article is marked with the same first trademark and the same second trademark.

5. The method of claim 1 wherein the second graphic is a seasonal graphic.

6. The method of claim 5 wherein the seasonal graphic relates to one of winter, spring, summer, or fall.

7. The method of claim 5 wherein the seasonal graphic relates to one of Valentine's Day, St. Patrick's Day, Easter, the 4^{th} of July, Halloween, Thanksgiving, Christmas, Hanukkah, or New Years Day.

8. The method of claim 1 wherein the first package includes information relating to the first disposable article and the second disposable article.

9. The method of claim 1 further comprising communicating with the consumer the limited availability of the second disposable article and the non-limited availability of the first disposable article.

10. The method of claim 1 wherein the first graphic includes a first character graphic and the second graphic includes a second character graphic, the first character graphic and the second character graphic depicting the same character.

11. The method of claim 1 wherein the first disposable article and the second disposable article differ only in graphics.

12. A method of providing an array of disposable absorbent articles, the array manufactured by or for the same business entity, comprising:
providing an array of at least three different disposable articles, the array comprising:
a first disposable article having a first outer cover; a first absorbent structure disposed on the first outer cover; and a first graphic disposed on the first outer cover; the first disposable article being enclosed in a first package;
a second disposable article having a second outer cover; a second absorbent structure disposed on the second outer cover; and a second graphic disposed on the second outer cover; the second disposable article being enclosed in a second package;
a third disposable article having a third outer cover; a third absorbent structure disposed on the third outer cover; and a third graphic disposed on the third outer cover; the third disposable article being enclosed in a third package;
wherein the first disposable article is provided during a first period of time, a second period of time, a third period of time, and a fourth period of time, the fourth period of time being after the third period of time, the third period of time being after the second period of time, and the second period of time being after the first period of time;
the second disposable article is provided during the second period of time, and the second disposable article is not provided during the first period of time, the third period of time, and the fourth period of time;
the third disposable article is provided during the third period of time, and the third disposable article is not provided during the first period of time, the second period of time, and the fourth period of time;
the first graphic differs from the second and third graphic, and the second graphic differs from the third graphic; and
the first absorbent structure, the second absorbent structure, and the third absorbent structure are substantially identical.

13. The method of claim 12 wherein the first package includes information relating to the first disposable article, the second disposable article, and the third disposable article.

14. The method of claim 12 wherein the first disposable article, the second disposable article, and the third disposable article differ only in graphics.

15. The method of claim 12 wherein the second graphic or the third graphic is a seasonal graphic.

16. The method of claim 15 wherein the seasonal graphic relates to one of winter, spring, summer, or fall.

17. The method of claim 15 wherein the seasonal graphic relates to one of Valentine's Day, St. Patrick's Day, Easter, the 4^{th} of July, Halloween, Thanksgiving, Christmas, Hanukkah, or New Years Day.

18. An array of disposable absorbent articles, the array manufactured by or for the same business entity, the array comprising:
a first disposable article having a first outer cover; a first absorbent structure disposed on the first outer cover; and a first graphic disposed on the first outer cover; the first disposable article being enclosed in a first package; and
a second disposable article having a second outer cover; a second absorbent structure disposed on the second outer cover; and a second graphic disposed on the second outer cover; the second disposable article being enclosed in a second package;
wherein the first disposable article is provided during a first period of time, a second period of time, and a third period of time, the third period of time being after the second period of time and the second period of time being after the first period of time;
the second disposable article is provided during the second period of time, and the second disposable article is not provided during the first period of time, and the third period of time; the first graphic differs from the second graphic; and
the first absorbent structure and the second absorbent structure are substantially identical.

19. The array of disposable diapers of claim 18 wherein the first package includes information relating to the first disposable article and the second disposable article.

20. The method of claim 1 wherein the first disposable article and the second disposable article differ only in graphics.
